(19) United States
(12) Patent Application Publication
(10) Pub. No.: US 2026/0108208 A1
(43) Pub. Date: Apr. 23, 2026

(54) SYSTEMS AND METHODS FOR IDENTIFYING AND TREATING NEUROLOGICAL DEFICIENCIES USING BRAIN IMAGING DATA

(71) Applicant: Matter Neuroscience Inc., Boulder, CO (US)

(72) Inventor: Axel BOUCHON, Kleinmachnow (DE)

(73) Assignee: Matter Neuroscience Inc., Boulder, CO (US)

(21) Appl. No.: 19/361,208

(22) Filed: Oct. 17, 2025

Related U.S. Application Data
(60) Provisional application No. 63/708,885, filed on Oct. 18, 2024.

(51) Int. Cl. A61B 5/00; A61B 5/055; A61B 5/16; G01R 33/48

(52) U.S. Cl. CPC A61B 5/486; A61B 5/0036; A61B 5/055; A61B 5/165; A61B 5/4064; A61B 5/742; G01R 33/4806

(57) ABSTRACT

Described are systems, methods, and non-transitory computer-readable storage media for treating a neurological deficiency in a patient. The method includes providing to the patient a plurality of stimuli based on a plurality of positive memories of the patient; while providing the plurality of stimuli, measuring brain activity of the patient by collecting data indicating one or more regions of activity in the brain and an associated intensity of brain activity in the one or more regions; and displaying to the patient a graphical visualization representative of a plurality of positive emotions. Each emotion can be assigned a color in the graphical visualization, and an intensity of the color in the graphical visualization can indicate an emotional intensity of a given emotion that is associated with the one or more stimuli.

### CROSS-REFERENCE TO RELATED APPLICATIONS

This application claims the benefit of U.S. Provisional Application No. 63/708,885, filed Oct. 18, 2024, the entire contents of which is incorporated herein by reference.

### FIELD

This disclosure relates generally to systems and methods for identifying and treating neurological deficiencies in a patient, and more specifically, to systems and methods for identifying and treating neurological deficiencies in a patient using brain imaging techniques and a graphical visualization depicting emotional intensities associated with stimuli provided to the patient.

### BACKGROUND

Mental health has become increasingly recognized as something that should be cared for with as much concern as physical health. Today, it is estimated that more than 80% of the worldwide population are burdened by depression, anxiety, or addiction at least once in their lives. This erosion of mental health has consequential implications for society at large. Mental health issues can be attributed to increases in crime, suicide rates, and even loss of productivity. Thus, improving mental health across a population not only confers benefits to the individual whose mental health is improved, but also benefits society.

Recognizing the importance of mental health, many approaches to improving mental health have been suggested and applied. For instance, philosophical, psychological, technical, and neuroscientific approaches to improving mental health have been implemented in isolation, focusing only on a single aspect of the disease (rather than holistically) with mixed success. Many of these approaches have failed or provided marginal improvements in mental health. The incidence and prevalence of depressive disorders have been skyrocketing in recent years with more than 260 million people impacted worldwide and a life-time incidence rate of >75%. Efficacy and effectiveness of available anti-depressants is historically very low with >70% of patients not experiencing any improvement or even progressing to a more severe disease state. The issue is further exacerbated by substantial deterioration of disease states after discontinuation of anti-depressants with significantly higher suicide rate observed in certain patient groups.

Basic research has provided evidence that depression is caused by one or more dysfunctional reward systems. However, a precise method to identify the molecular basis for targeted intervention and leverage it for individualized therapy is missing. Thus, available pharmacological and behavioral therapies can't be properly matched to the underlying cause of disease, leading to inefficient therapies. There is a substantial risk that the treatments approved and used today are actually making the disease worse over time since often times, conventional treatments are not properly addressing the underlying molecular impairment.

### SUMMARY

Disclosed herein are systems and methods for identifying neurological disorders, visually representing emotional and neurological activity, and evaluating and treating depressive disorders based on the emotional and neurological information. The disclosed systems and methods integrate subjective emotional data, such as photographs representing a subject's peak memories, with functional magnetic resonance imaging (fMRI) data to identify and analyze brain regions associated with positive emotional responses. The system generates a visual representation that displays emotional activation states using varying color intensities corresponding to neural activity levels within regions of interest (ROIs). This visual representation enables real-time neurofeedback during treatment sessions, allowing subjects to observe and modulate their emotional responses by engaging more deeply with effective memories. Through repeated visualization and neurofeedback training, the subject can enhance control over emotional regulation, contributing to personalized treatment outcomes for depressive disorders.

In some aspects, systems, methods, and non-transitory computer-readable storage media storing instructions are provided, the instructions including the method for identifying and treating a neurological deficiency in a patient, the method performed by one or more processors of the system, the method comprising: receiving memory information associated with a patient that includes a plurality of patient memories representative of a plurality of positive emotions and a plurality of emotional intensities associated with the plurality of patient memories; determining one or more neurotransmitter levels of the patient based at least on the plurality of emotional intensities, wherein the determined one or more neurotransmitter levels are indicative of the neurological deficiency; and treating the neurological deficiency in accordance with a treatment regimen, the treatment regimen comprising: (i) providing a plurality of stimuli based on the plurality of patient memories to the patient; (ii) while providing the plurality of stimuli, measuring brain activity of the patient, wherein measuring brain activity comprises collecting data indicating one or more regions of activity in the brain and an associated intensity of brain activity in the one or more regions; (iii) providing to the patient a first exposure to one or more of the plurality of stimuli based on the data collected in response to the one or more of the plurality of stimuli; (iv) displaying to the patient a graphical visualization representative of the plurality of emotions, wherein each emotion is assigned a color, and an intensity of the color in the graphical visualization indicates an emotional intensity of a given emotion that is associated with the one or more stimuli; (v) repeating steps (iii) and (iv) for a second exposure to the one or more stimuli, the second exposure subsequent to the first exposure; (vi) providing the one or more stimuli to the patient intermittently for a first period; and (vii) repeating steps (i) through (vi) for a second period to treat the neurological deficiency of the patient.

In some aspects, systems, methods, and non-transitory computer-readable storage media storing instructions are provided, the instructions including the method for treating a neurological deficiency in a patient, the method performed by one or more processors of the system, the method comprising: providing to the patient a plurality of stimuli based on a plurality of positive memories of the patient; while providing the plurality of stimuli, measuring brain activity of the patient by collecting data indicating one or more regions of activity in the brain and an associated intensity of brain activity in the one or more regions; and displaying to the patient a graphical visualization representative of a plurality of positive emotions, wherein each emotion is assigned a color, and an intensity of the color in the graphical visualization indicates an emotional intensity of a given emotion that is associated with the one or more stimuli.

### BRIEF DESCRIPTION OF THE FIGURES

Illustrative aspects of the present disclosure are described in detail below with reference to the following figures. It is intended that the embodiments and figures disclosed herein are to be considered illustrative and exemplary rather than restrictive.

FIG. 1A illustrates an exemplary method for identifying and treating a neurological deficiency in a patient, according to some embodiments.

FIG. 1B illustrates an exemplary treatment regimen for treating a neurological deficiency in accordance with the method shown in FIG. 1A, in accordance with some embodiments.

FIG. 2A illustrates an initial emotion neurofeedback wheel prior to use, in accordance with some embodiments.

FIG. 2B illustrates the emotion neurofeedback wheel shown in FIG. 2A during an exemplary use case, in accordance with some embodiments.

FIG. 3 illustrates an exemplary computing device, in accordance with some embodiments.

### DETAILED DESCRIPTION

Disclosed herein are systems, methods, and non-transitory computer readable storage media storing instructions for evaluating the underlying molecular cause for a depressive disorder and treatment plans based thereon. The systems can utilize brain imaging techniques, subjective emotion data taken from a subject, and/or one or more algorithms for translating emotional states to neurotransmitters to determine the molecular cause for a depressive disorder of a patient and to treat the depressive disorder. The subjective emotion data can be based on one or more peak memories of the subject, represented by a photo, that is associated with one of the nine positive emotions. The subjective emotion data can include an emotional intensity associated with each memory. The subjective emotion data can be used as input to an algorithm (e.g., a positive emotion-to-neurotransmitter or PE-NT matrix) to determine the underlying neurotransmitter levels. The subject may also identify control stimuli that includes one or more control photos that do not trigger any emotion and/or are not associated with a peak memory.

The subject may undergo a functional magnetic resonance imaging (fMRI) session in which the subject is randomly exposed to control stimuli or peak memories. The induced brain activity while observing the control stimuli and peak memories can be assessed in real time to determine one or more regions-of-interest (ROIs) in the brain representative of the greatest amount of activity triggered by any of the peak memories and/or the greatest amount of activity for one or more of the nine positive emotions. Following the fMRI session, the subject may be exposed to one or more of the most effective memories determined with respect to the ROI activation. The subject may observe a visual representation of the emotions with varying color intensities corresponding to their activation state in the ROI. The subject may intensify the colors on the visual representation by immersing deeper with the effective memories.

Following the neurofeedback training session, the subject may undergo a series of home training sessions involving memory recall. The subject may undergo several sessions of neurofeedback training and home training sessions for a treatment totaling four fMRI sessions over a period of about two months. One or more clinical assessments of the emotional stability and depression relapse of the subject can be performed following the treatment.

In the following description of the disclosure and embodiments, reference is made to the accompanying drawings in which are shown, by way of illustration, specific embodiments that can be practiced. It is to be understood that other embodiments and examples can be practiced, and changes can be made, without departing from the scope of the disclosure.

In addition, it is also to be understood that the singular forms “a,” “an,” and “the” used in the following description are intended to include the plural forms as well unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic, or optical signals capable of being stored, transferred, combined, compared, and otherwise manipulated. It is convenient at times, principally for reasons of common usage, to refer to these signals as bits, values, elements, symbols, characters, terms, numbers, or the like. Furthermore, it is also convenient at times to refer to certain arrangements of steps requiring physical manipulations of physical quantities as modules or code devices without loss of generality.

However, all of these and similar terms are to be associated with the appropriate physical quantities and are merely convenient labels applied to these quantities. Unless specifically stated otherwise as apparent from the following discussion, it is appreciated that, throughout the description, discussions utilizing terms such as “processing,” “computing,” “calculating,” “determining,” “displaying,” or the like refer to the action and processes of a computer system, or similar electronic computing device, that manipulates and transforms data represented as physical (electronic) quantities within the computer system memories or registers or other such information storage, transmission, or display devices.

Certain aspects of the present Disclosure include process steps and instructions described herein in the form of an algorithm. It should be noted that the process steps and instructions of the present Disclosure could be embodied in software, firmware, or hardware, and, when embodied in software, they could be downloaded to reside on and be operated from different platforms used by a variety of operating systems.

The present disclosure also relates to a device for performing the operations herein. This device may be specially constructed for the required purposes, or it may comprise a general-purpose computer selectively activated or reconfigured by a computer program stored in the computer. Such a computer program may be stored in a non-transitory, computer-readable storage medium such as, but not limited to, any type of disk, including floppy disks, optical disks, CD-ROMs, magnetic-optical disks, read-only memories (ROMs), random access memories (RAMs), EPROMs, EEPROMs, magnetic or optical cards, application-specific integrated circuits (ASICs), or any type of media suitable for storing electronic instructions and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present disclosure is not described with reference to any particular programming language. It will be appreciated that a variety of programming languages may be used to implement the teachings of the present disclosure as described herein.

Depression or other neurological disorders can often be attributed to dysfunctional reward systems in the brain of a patient, wherein the brain is often failing to produce adequate brain reward system molecules associated with stable moods. Often times, anti-depressant medication is prescribed to a patient to treat a mood disorder, however such treatments often don't provide satisfactory outcomes, with patients often not experiencing any improvement in their condition, and in some cases exacerbating their condition when they eventually discontinue the medication. While it is known that lack of certain brain reward system molecules can be at the root cause of certain mood disorders, precisely identifying which neurotransmitter/molecule is deficient, and then using such knowledge to generate a treatment plan for the patient is lacking. This deficiency means that behavior and pharmacological therapies to treat mood disorders can't be matched to the underlying cause of the mood disorder, thus leading to poorer outcomes for patients with the mood disorder.

As described above, the first step in generating effective therapies (behavioral and pharmacological) for the treatment of mood disorders can be to accurately identify any deficiencies in the reward molecules that may serve as the underlying cause of the mood disorder. For instance, mood disorders (e.g., depression) can often be linked to deficiencies in the brain of various reward molecules including but not limited to dopamine, serotonin, testosterone, oxytocin, cannabinoids, and opioids. Detecting deficiencies in these reward molecules can be challenging. Thus, and as described in detail below, a method for detecting deficiencies in reward molecules is presented that can combine various diagnostic tools to accurately detect deficiencies in reward molecules.

### Mood Inventory and Biomarker Measures

Prior to neurofeedback training, patients can be clinically assessed for depression disorders, (e.g., by the Montgomery and Asberg Depression Rating Scale (MADRS) depression scale), mood inventory (e.g., by the profile of mood states (POMS) scale), and/or by a broad spectrum of blood biomarkers checking key hormonal markers for mood disorders (e.g., Vitamin D3, Cortisol, Testosterone, Oxytocin, etc.). Changes in these clinical and molecular markers can represent improvement in the treatment described herein. Patients with a major depressive disorder represented by a MADRS scale of >34 may be excluded given the complexity of the procedure.

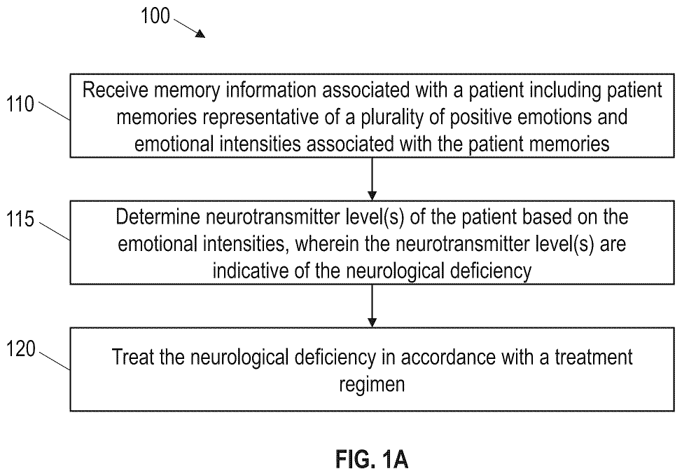

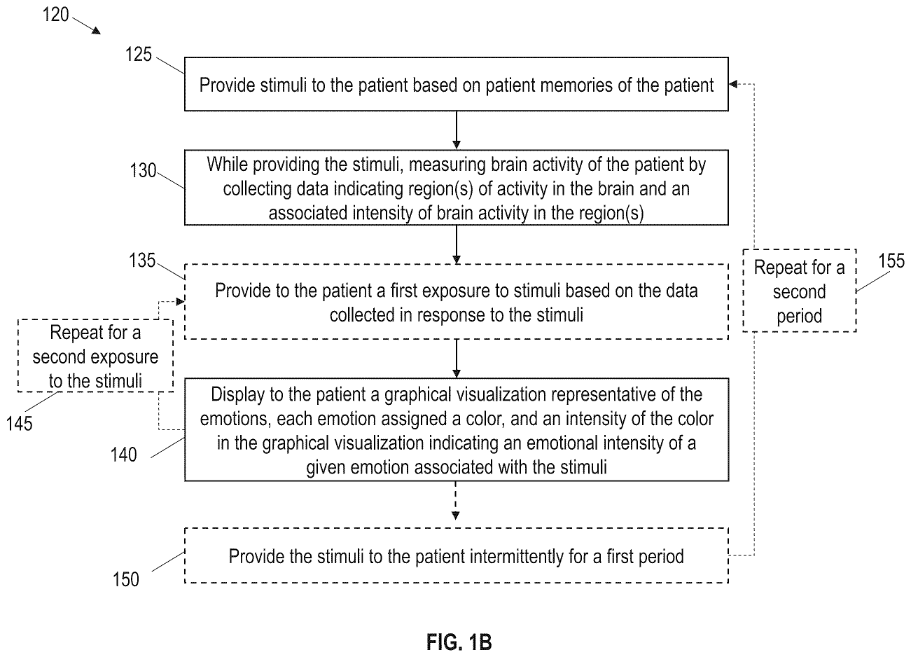

Once the clinical assessment is complete, neurofeedback training may begin. FIG. 1A illustrates a method 100 for identifying and treating a neurological deficiency in a patient. At block 110, the method 100 can include receiving memory information associated with a patient. The memory information may include patient memories representative of a plurality of positive emotions and emotional intensities associated with the patient memories.

In some embodiments, the patient may be asked to identify one or more autobiographic peak memories primarily represented by one of the nine emotions for each of the emotions. A peak memory can be defined as a positive memory that is among the top 1% of all memories of the patient. In some embodiments, the memory information includes one or more photos, videos, and/or audio that is representative of the patient memories.

In some embodiments, a given patient memory is associated with more than one of the positive emotions. Thus, receiving memory information can include receiving a respective emotional intensity for each of the one or more emotions associated with the given patient memory. The emotions may include, but are not limited to, enthusiasm/motivation/excitement, sexual desire, pride/recognition/self-esteem, nurturant love/family love, contentment, friendship love/amusement, pleasure, and gratitude. The patient may be asked to identify and enter a representative photo or video for each the peak memories into a mobile application (otherwise referred to herein as an app) and to assess the emotional intensities associated with each memory in the app. The patient may be asked to assess the emotional intensity of these emotions, for example, in the instance more than one emotion is associated with a given memory. The emotion assessment can then be used to calculate the underlying neurotransmitter levels of the patient with an algorithm, such as a positive emotion-to-neurotransmitter (PE-NT) matrix. Exemplary neurotransmitters may include, but are not limited to, dopamine, serotonin, testosterone, oxytocin, cannabinoids, and opioids. An exemplary PE-NT matrix is described in greater detail in U.S. Pat. No. 12,207,927, the contents of which are incorporated herein in its entirety.

In some examples, the treatment may utilize control stimuli to assess the emotions of the patient. Thus, the patient may identify one or more control photos that do not trigger any emotion and/or are not associated with an autobiographic memory that can serve as control stimuli. Accordingly, in some examples, receiving memory information may include receiving one or more neutral patient memories that do not evoke any of the plurality of positive emotions.

At block 115, the method 100 can include determining one or more neurotransmitter levels of the patient based on the emotional intensities. The one or more neurotransmitter levels can be indicative of the neurological deficiency. Determining neurotransmitter levels can include applying one or more algorithms configured to convert a patient's recorded memories and emotion data into neurotransmitter transmitter levels (e.g., reward system molecules), thus allowing for the identification of neurotransmitter deficiencies. The algorithms may be configured input a patient's memories, translate the memories into one or more neurotransmitter levels, and identify one or more deficiencies in neurotransmitter levels.

In some examples, determining neurotransmitter levels based on the emotional intensities is based at least in part on locations within the brain for which a signal is to be measured in response to stimuli, and signal intensities based on measured brain activity of the patient. Thus, the method 100 can include (at block 115) determining one or more locations within the brain for which a signal is to be measured in response to the stimuli, and determining one or more signal intensities based on measured brain activity of the patient, the signal intensities associated with each of the one or more locations of the brain. In some examples, determining the neurotransmitter levels can include comparing a determined signal intensity for a determined location to a threshold signal intensity level for that location. In some examples, the stimuli presented to the patient are analyzed based on the emotion(s) associated with the memories that are associated with the stimuli, as indicated in the received memory information. In this example, determining the locations within the brain can be based at least in part on the one or more emotions indicated in the received memory information.

Exemplary systems and methods for evaluating neurological deficiencies using brain scan data are described in greater detail in Patent Cooperation Treaty (PCT) Application No. PCT/US2023/071174, the contents of which are incorporated herein in its entirety. For example, one or more aspects of the systems and methods described in PCT/US2023/071174 may be used in conjunction with, in place of, or in addition to one or more aspects of the treatment plan described herein.

At block 120, the method 100 can include treating the neurological deficiency in accordance with a treatment regimen. FIG. 1B illustrates an exemplary treatment regimen for treating a neurological deficiency in accordance with the method 100 shown in FIG. 1A. The treatment regimen shown in FIG. 1B is described in greater detail below with respect to exemplary neurofeedback training.

### Neurofeedback Training

After completing the initial mood inventory and biomarker assessment, the patient may undergo the first magnetic resonance imaging (e.g., fMRI, such as 7 T fMRI) session, otherwise referred to herein as the localizer session. At block 125, the method 100 can include providing a plurality of stimuli based on the plurality of patient memories to the patient. In some embodiments, providing the plurality of stimuli includes randomly providing one or more control stimuli based on the one or more neutral patient memories. For example, in the localizer session, the patient may be randomly exposed to control stimuli or peak memories, and the induced brain activity while observing the memory photo may be assessed in real-time.

At block 130, the method 100 can include measuring brain activity of the patient while providing the plurality of stimuli. In some examples, measuring brain activity includes collecting data indicating one or more regions of activity in the brain and an associated intensity of brain activity in the one or more regions. Deficiencies of neurotransmitter levels within a patient's brain may be determined by measuring brain activity using brain imaging techniques, such as functional magnetic resonance imaging (fMRI) and/or other similar techniques. For example, the patient's brain can be imaged in real-time while the patient is being randomly exposed to photos and/or music that have emotional significance to the patient and thus are likely to cause reward center activity in the brain of the patient. The signal intensity data from the brain scan and/or 3D location of brain activity can be used to identify distinct regions of interest in the brain that can be correlated with distinct emotions and/or reward system molecules (e.g., neurotransmitters).

In some examples, measuring the brain activity includes a 7 Tesla (T) functional magnetic resonance imaging (fMRI) session. Alternatively, measuring the brain activity includes an electroencephalogram (EEG) scan, or functional near-infrared spectroscopy (fNIRs).

In some examples, the observing physician can localize a region-of-interest (ROI) representative of the greatest activity triggered by any of the peak memory based on the observed brain stimulation. In addition, the physician may select the ROI(s) that show the greatest activity in areas representative of distinct emotions. These ROIs may have been identified in previous studies. Particularly high or low activity changes in these emotion-specific ROIs may provide insights in the underlying cause for the depression. For example, if an ROI known to be associated with friendship love and cannabinoids is low in activation upon stimulation with a memory selected for friendship love, it may be indicative of an underlying issue of the clinical manifestation of the depressive disorder, namely a lack of belonging and friends. The ROI representing the maximum activation by any memory can be different from the ROI for each of the distinct emotions.

At block 135, the method 100 can include providing to the patient a first exposure to one or more of the plurality of stimuli based on the data collected in response to the one or more of the plurality of stimuli. For example, after the localizer session, the patient may be exposed to the most effective one or more memories with respect to ROI activation localized during the localizer session. The patient may be exposed to the photo representing and triggering the peak memory recall for an initial period (e.g., less than 30 seconds) and may be asked to emotionally immerse with the memory for this period.

After this initial period, at block 140, the method 100 can include displaying to the patient a graphical visualization representative of the plurality of emotions. In some embodiments, one or more of the emotions is assigned a color. An intensity of the color in the graphical visualization can indicate an emotional intensity of a given emotion that is associated with the one or more stimuli. The graphical visualization may correspond and be linked in real-time to a region of activity in the brain. In this way, the graphical visualization may automatically update based on the emotional intensity of the given emotion associated with the one or more stimuli. For example, the graphical visualization can be linked in real-time to the ROI and may be representative of the activation state in the ROI. Such a visualization can be a colored graph, thermometer, or a wheel with different color intensities.

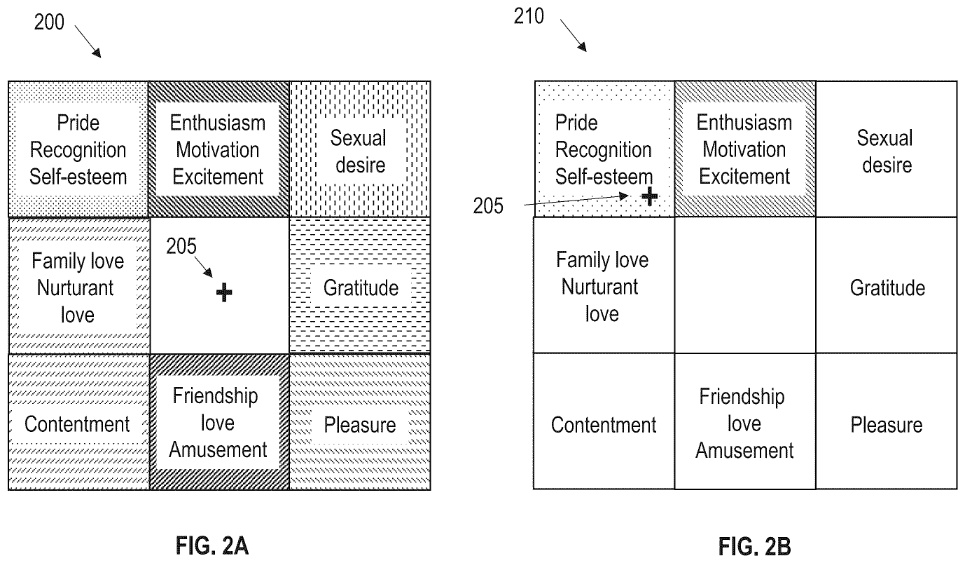

FIG. 2A illustrates an initial emotion neurofeedback wheel 200 prior to use. The emotion neurofeedback wheel can include different colors for each of the emotions. The intensity of the colors can represent the strength of the engagement in the brain ROI. The emotion neurofeedback wheel 200 can include a cursor 205 that indicates which emotion is engaged or being experienced.

Returning to method 100, at block 145, the method 100 can include repeating blocks 135 and 140 to provide a second exposure to one or more of the stimuli. The second exposure may be subsequent to the first exposure described above with respect to block 135. In some examples, providing the second exposure to one or more of the plurality of stimuli to the patient may include providing a second exposure to the stimuli that evoke the greatest measured brain activity amongst the plurality of stimuli. In some examples, providing the one or more stimuli to the patient at block 145 occurs while displaying the graphical visualization (e.g., neurofeedback wheel 200) and/or subsequent to displaying the graphical visualization. For example, the patient can be asked to increase the temperature on a thermometer or to intensify the colors on the color wheel 200 in a second exposure to the memory by immersing deeper into the memory in order to intensify the emotional experience. After such a second exposure the graphical visualization can be shown again.

FIG. 2B illustrates the emotion neurofeedback wheel shown in FIG. 2A during an exemplary use case. In this example, the intensity of the color in the wheel 210 for each of pride/recognition/self-esteem and enthusiasm/motivation/excitement is about half (50%) of that in the wheel 200 illustrated in FIG. 2A. The cursor 205 can automatically move to the area representing the emotions of pride/enthusiasm due to a higher activation of the ROIs connected with pride.

Returning to method 100, after the second exposure, the second round of neurofeedback training ends, and at block 150, the method 100 can include providing the one or more stimuli to the patient intermittently for a first period. Providing the one or more stimuli to the patient intermittently for the first period can include providing the one or more stimuli to the patient at home, every three days for a period of two weeks. For example, the patient can be sent home with the task to repeat the immersion into the memory every couple (e.g., three) days for a period of about two weeks (otherwise referred to herein as home training). The patient can use a memory recall functionality in the mobile app for home training.

At block 155, the method 100 can optionally include repeating blocks 125, 130, 135, 140, 145, and 150 for a second period to treat the neurological deficiency of the patient. This second period is discussed below with respect to an exemplary treatment plan.

### Treatment Plan

The treatment regimen may include four fMRI sessions. Each fMRI session may be spaced apart by a two-week at-home treatment session. For example, the MRI (e.g., 7 T fMRI) neurofeedback session can be repeated about two weeks after the first neurofeedback session. In addition, the clinical assessment, mood inventory and biomarker measures can be repeated at this time. The localized ROI from the first sessions can be used as an orientation. After the second fMRI neurofeedback session, the home training can again be repeated for the following two weeks.

The same procedure can be repeated for a third and a fourth round totaling four fMRI neurofeedback sessions and an entire study period of about two months (or eight weeks). After the final period of home training, the clinical assessment, mood inventory, and biomarker measures can be repeated to assess treatment outcomes.

The patient can be invited to follow-up assessments, including performing a clinical assessment, mood inventory, and biomarker measures. For example, the follow-up assessments may occur after one month, three months, and/or nine months to assess depression relapse and emotional stability of the patient.

### Exemplary Computing Systems

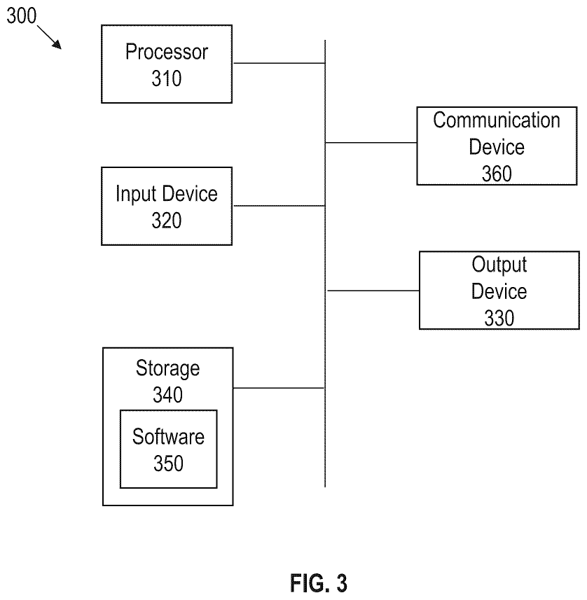

FIG. 3 illustrates an example of a computing device for identifying and treating a neurological deficiency in a patient. Device 300 can be a host computer connected to a network. Device 300 can be a client computer or a server. As shown in FIG. 3, device 300 can be any suitable type of microprocessor-based device, such as a personal computer, workstation, server, or handheld computing device (portable electronic device) such as a phone or tablet. The device can include, for example, one or more of processor 310, input device 320, output device 330, storage 340, and communication device 360. Input device 320 and output device 330 can generally correspond to those described above and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage, such as an electrical, magnetic, or optical memory, including a RAM, cache, hard drive, or removable storage disk. Communication device 360 can include any suitable device capable of transmitting and receiving signals over a network, such as a network interface chip or device. The components of the computer can be connected in any suitable manner, such as via a physical bus or wirelessly.

Software 350, which can be stored in storage 340 and executed by processor 310, can include, for example, the programming that embodies the functionality of the present disclosure (e.g., as embodied in the devices as described above).

Software 350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 340, that can contain or store programming for use by or in connection with an instruction execution system, apparatus, or device.

Software 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can implement any operating system suitable for operating on the network. Software 350 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### Exemplary Embodiments

The following embodiments are merely exemplary and are not intended to limit the scope of the disclosure herein.

Embodiment 1. A method for identifying and treating a neurological deficiency in a patient, the method performed by a system comprising one or more processors, the method comprising: receiving memory information associated with a patient that includes a plurality of patient memories representative of a plurality of positive emotions and a plurality of emotional intensities associated with the plurality of patient memories; determining one or more neurotransmitter levels of the patient based at least on the plurality of emotional intensities, wherein the determined one or more neurotransmitter levels are indicative of the neurological deficiency; and treating the neurological deficiency in accordance with a treatment regimen, the treatment regimen comprising: (i) providing a plurality of stimuli based on the plurality of patient memories to the patient; (ii) while providing the plurality of stimuli, measuring brain activity of the patient, wherein measuring brain activity comprises collecting data indicating one or more regions of activity in the brain and an associated intensity of brain activity in the one or more regions; (iii) providing to the patient a first exposure to one or more of the plurality of stimuli based on the data collected in response to the one or more of the plurality of stimuli; (iv) displaying to the patient a graphical visualization representative of the plurality of emotions, wherein each emotion is assigned a color, and an intensity of the color in the graphical visualization indicates an emotional intensity of a given emotion that is associated with the one or more stimuli; (v) repeating steps (iii) and (iv) for a second exposure to the one or more stimuli, the second exposure subsequent to the first exposure; (vi) providing the one or more stimuli to the patient intermittently for a first period; and (vii) repeating steps (i) through (vi) for a second period to treat the neurological deficiency of the patient.

Embodiment 2. The method of embodiment 1, wherein the memory information comprises photos, videos, and/or audio representative of the plurality of patient memories.

Embodiment 3. The method of embodiment 1 or 2, wherein a given patient memory is associated with more than one of the plurality of positive emotions, such that receiving the memory information comprises receiving a respective emotional intensity for each of the one or more emotions associated with the given patient memory.

Embodiment 4. The method of any one of embodiments 1-3, wherein receiving memory information comprises receiving one or more neutral patient memories that do not evoke any of the plurality of positive emotions, such that providing the plurality of stimuli at step (i) comprises randomly providing one or more control stimuli based on the one or more neutral patient memories.

Embodiment 5. The method of any one of embodiments 1-4, wherein providing the second exposure to one or more of the plurality of stimuli to the patient at step (iii) comprises providing a second exposure to the one or more stimuli that evoke the greatest measured brain activity amongst the plurality of stimuli.

Embodiment 6. The method of any one of embodiments 1-5, wherein the graphical visualization corresponds and is linked in real-time to a region of activity in the brain, such that the graphical visualization automatically updates based on the emotional intensity of the given emotion associated with the one or more stimuli.

Embodiment 7. The method of any one of embodiments 1-6, wherein the second exposure in step (v) comprises providing the one or more stimuli to the patient while displaying the graphical visualization and/or subsequent to displaying the graphical visualization.

Embodiment 8. The method of any one of embodiments 1-7, wherein providing the one or more stimuli to the patient intermittently for the first period in step (vi) comprises providing the one or more stimuli to the patient at home, every three days for a period of two weeks.

Embodiment 9. The method of any one of embodiments 1-8, wherein measuring the brain activity comprises a 7 Tesla (T) functional magnetic resonance imaging (fMRI) session.

Embodiment 10. The method of any one of embodiments 1-9, wherein the treatment regimen comprises four fMRI sessions, each fMRI session spaced apart by a two-week at-home treatment session.

Embodiment 11. A system comprising one or more processors and memory storing instructions configured to be executed by the one or more processors to cause the system to: receive memory information associated with a patient that includes a plurality of patient memories representative of a plurality of positive emotions and a plurality of emotional intensities associated with the plurality of patient memories; determine one or more neurotransmitter levels of the patient based at least on the plurality of emotional intensities, wherein the determined one or more neurotransmitter levels are indicative of the neurological deficiency; and treat the neurological deficiency in accordance with a treatment regimen, the treatment regimen comprising: (i) providing a plurality of stimuli based on the plurality of patient memories to the patient; (ii) while providing the plurality of stimuli, measuring brain activity of the patient, wherein measuring brain activity comprises collecting data indicating one or more regions of activity in the brain and an associated intensity of brain activity in the one or more regions; (iii) providing to the patient a first exposure to one or more of the plurality of stimuli based on the data collected in response to the one or more of the plurality of stimuli; (iv) displaying to the patient a graphical visualization representative of the plurality of emotions, wherein each emotion is assigned a color, and an intensity of the color in the graphical visualization indicates an emotional intensity of a given emotion that is associated with the one or more stimuli; (v) repeating steps (iii) and (iv) for a second exposure to the one or more stimuli, the second exposure subsequent to the first exposure; (vi) providing the one or more stimuli to the patient intermittently for a first period; and (vii) repeating steps (i) through (vi) for a second period to treat the neurological deficiency of the patient.

Embodiment 12. A non-transitory computer-readable storage medium storing instructions which, when executed by a system comprising one or more processors, cause the system to: receive memory information associated with a patient that includes a plurality of patient memories representative of a plurality of positive emotions and a plurality of emotional intensities associated with the plurality of patient memories; determine one or more neurotransmitter levels of the patient based at least on the plurality of emotional intensities, wherein the determined one or more neurotransmitter levels are indicative of the neurological deficiency; and treat the neurological deficiency in accordance with a treatment regimen, the treatment regimen comprising: (i) providing a plurality of stimuli based on the plurality of patient memories to the patient; (ii) while providing the plurality of stimuli, measuring brain activity of the patient, wherein measuring brain activity comprises collecting data indicating one or more regions of activity in the brain and an associated intensity of brain activity in the one or more regions; (iii) providing to the patient a first exposure to one or more of the plurality of stimuli based on the data collected in response to the one or more of the plurality of stimuli; (iv) displaying to the patient a graphical visualization representative of the plurality of emotions, wherein each emotion is assigned a color, and an intensity of the color in the graphical visualization indicates an emotional intensity of a given emotion that is associated with the one or more stimuli; (v) repeating steps (iii) and (iv) for a second exposure to the one or more stimuli, the second exposure subsequent to the first exposure; (vi) providing the one or more stimuli to the patient intermittently for a first period; and (vii) repeating steps (i) through (vi) for a second period to treat the neurological deficiency of the patient.

Embodiment 13. A method for treating a neurological deficiency in a patient, the method performed by a system comprising one or more processors, the method comprising: providing to the patient a plurality of stimuli based on a plurality of positive memories of the patient; while providing the plurality of stimuli, measuring brain activity of the patient by collecting data indicating one or more regions of activity in the brain and an associated intensity of brain activity in the one or more regions; and displaying to the patient a graphical visualization representative of a plurality of positive emotions, wherein each emotion is assigned a color, and an intensity of the color in the graphical visualization indicates an emotional intensity of a given emotion that is associated with the one or more stimuli.

Embodiment 14. The method of embodiment 13, wherein the graphical visualization corresponds and is linked in real-time to a region of activity in the brain, such that the graphical visualization automatically updates based on the emotional intensity of the given emotion associated with the one or more stimuli.

Embodiment 15. The method of embodiment 13, wherein providing the plurality of stimuli comprises randomly providing one or more control stimuli based on one or more neutral patient memories.

Embodiment 16. The method of embodiment 13, wherein measuring the brain activity comprises a 7 Tesla (T) functional magnetic resonance imaging (fMRI) session.

Embodiment 17. The method of embodiment 13, comprising further providing one or more of the plurality of stimuli to the patient while displaying the graphical visualization and/or subsequent to displaying the graphical visualization.

Embodiment 18. The method of embodiment 13, wherein further providing the one or more of the plurality of stimuli to the patient comprises providing the one or more stimuli that evoke the greatest measured brain activity amongst the plurality of stimuli.

Embodiment 19. A system comprising one or more processors and memory storing instructions configured to be executed by the one or more processors to cause the system to: provide to the patient a plurality of stimuli based on a plurality of positive memories of the patient; while providing the plurality of stimuli, measure brain activity of the patient by collecting data indicating one or more regions of activity in the brain and an associated intensity of brain activity in the one or more regions; and display to the patient a graphical visualization representative of a plurality of positive emotions, wherein each emotion is assigned a color, and an intensity of the color in the graphical visualization indicates an emotional intensity of a given emotion that is associated with the one or more stimuli.

Embodiment 20. A non-transitory computer-readable storage medium storing instructions which, when executed by a system comprising one or more processors, cause the system to: provide to the patient a plurality of stimuli based on a plurality of positive memories of the patient; while providing the plurality of stimuli, measure brain activity of the patient by collecting data indicating one or more regions of activity in the brain and an associated intensity of brain activity in the one or more regions; and display to the patient a graphical visualization representative of a plurality of positive emotions, wherein each emotion is assigned a color, and an intensity of the color in the graphical visualization indicates an emotional intensity of a given emotion that is associated with the one or more stimuli.

### EXAMPLES

### Example 1: Study of Neurofeedback & Home Training

Subjects are clinically assessed for depression disorders, (e.g., by the MADRS depression scale), mood inventory (e.g., by the POMS scale), and by a broad spectrum of blood biomarkers checking key hormonal markers for mood disorders (e.g., Vitamin D3, Cortisol, Testosterone, Oxytocin). Changes in these clinical and molecular markers represent improvement in the procedure. Subjects with a major depressive disorder represented by a MADRS scale of >34 are excluded given the complexity of the procedure.

The subjects are educated to the neuroscience of happiness, emotions, and memory formation and can be introduced to the nine positive emotions (enthusiasm, sexual desire, pride/recognition, nurturant love, contentment, amusement, attachment love, pleasure, and gratitude) and six underlying neurotransmitters (dopamine, testosterone, serotonin, oxytocin, cannabinoids, and opioids) selected by evolution. The procedure is explained in detail at this stage.

Thereafter the subject is asked to identify three autobiographic peak memories primarily represented by one of the nine emotions. A peak memory is defined as a positive memory that is among the top 1% of all memories of the subject. The subject is then asked to identify and enter a representative photo for each the 27 peak memories into the Matter app and assess all emotional intensities associated with each memory in the app. In the instance more than one emotion is associated with a memory, the subject is asked to also assess the emotional intensity of these emotions. The emotion assessment is then used to calculate the underlying neurotransmitter levels with the PE-NT-Matrix.

In order to have representative control stimuli, the subject is asked next to identify 12 control photos that do not trigger any emotion and are not associated with an autobiographic memory.

Education, peak memory, and control stimuli selection is completed within 48 h. Thereafter, the subject is invited to the first 7 T fMRI session, otherwise referred to as the localizer session. In the localizer session the subject is randomly exposed to control stimuli or peak memories and the induced brain activity while observing the memory photo is assessed in real-time. Based on the observed brain stimulation, the observing physician is localizing a region-of-interest (ROI) that represents the highest activity triggered by any of the peak memory. In addition, the physician is selecting the ROIs that show the highest activity in areas representative for distinct emotions. Particularly high or low activity changes in these emotion-specific ROIs may provide insights in the underlying cause for the depression.

After the localizer session, the subject is exposed to the most effective one or two memories in respect to ROI activation localized during the localizer session. The subject is exposed for 15-20 seconds to the photo representing and triggering the peak memory recall and is asked to emotionally immerse with the memory for these 15-20 seconds. After this initial period, the subject is shown a simple visual graph that is linked in real-time to the ROI and represents the activation state in the ROI. Such a visual can be a thermometer or a color wheel with different color intensities (e.g., as shown in FIGS. 2A-2B). The subject is then asked to increase the temperature on the thermometer or to intensify the colors on the color wheel in a second exposure to the memory by immersing deeper into the memory in order to intensify the emotional experience. After such a second exposure of 15-20 seconds the visual graph is shown again. After the second exposure, the second round of neurofeedback training this session ends.

The subject is sent home with the task to repeat the immersion into the memory every three days for the next two weeks (“home training”). The subject uses a memory recall functionality in the Matter app for home training.

The next 7 T fMRI neurofeedback session occurs two weeks after the first neurofeedback session. In addition, the clinical assessment, mood inventory and biomarker measures are repeated. The localized ROI from the first sessions is used again as an orientation. After the second 7 T fMRI neurofeedback session the home training is again repeated for the following two weeks.

The same procedure is repeated for a third and a fourth round totaling the fMRI neurofeedback sessions to four and the entire study period to 8 weeks. After the final two weeks of home training the clinical assessment, mood inventory and biomarker measures are repeated.

The subjects are invited to follow-up assessments—again performing clinical assessment, mood inventory and biomarker measures—after one month, three months and nine months in order to assess depression relapse and emotional stability of the subject.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

This application discloses several numerical ranges in the text and figures. The numerical ranges disclosed inherently support any range or value within the disclosed numerical ranges, including the endpoints, even though a precise range limitation is not stated verbatim in the specification, because this disclosure can be practiced throughout the disclosed numerical ranges.

The above description is presented to enable a person skilled in the art to make and use the disclosure, and it is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded the widest scope consistent with the principles and features disclosed herein. Finally, the entire disclosure of the patents and publications referred in this application are hereby incorporated herein by reference.

What is claimed is:

1. A method for identifying and treating a neurological deficiency in a patient, the method performed by a system comprising one or more processors, the method comprising:
receiving memory information associated with a patient that includes a plurality of patient memories representative of a plurality of positive emotions and a plurality of emotional intensities associated with the plurality of patient memories;
determining one or more neurotransmitter levels of the patient based at least on the plurality of emotional intensities, wherein the determined one or more neurotransmitter levels are indicative of the neurological deficiency; and
treating the neurological deficiency in accordance with a treatment regimen, the treatment regimen comprising:
(i) providing a plurality of stimuli based on the plurality of patient memories to the patient;
(ii) while providing the plurality of stimuli, measuring brain activity of the patient, wherein measuring brain activity comprises collecting data indicating one or more regions of activity in the brain and an associated intensity of brain activity in the one or more regions;
(iii) providing to the patient a first exposure to one or more of the plurality of stimuli based on the data collected in response to the one or more of the plurality of stimuli;
(iv) displaying to the patient a graphical visualization representative of the plurality of emotions, wherein each emotion is assigned a color, and an intensity of the color in the graphical visualization indicates an emotional intensity of a given emotion that is associated with the one or more stimuli;
(v) repeating steps (iii) and (iv) for a second exposure to the one or more stimuli, the second exposure subsequent to the first exposure;
(vi) providing the one or more stimuli to the patient intermittently for a first period; and
(vii) repeating steps (i) through (vi) for a second period to treat the neurological deficiency of the patient.

2. The method of claim 1, wherein the memory information comprises photos, videos, and/or audio representative of the plurality of patient memories.

3. The method of claim 1, wherein a given patient memory is associated with more than one of the plurality of positive emotions, such that receiving the memory information comprises receiving a respective emotional intensity for each of the one or more emotions associated with the given patient memory.

4. The method of claim 1, wherein receiving memory information comprises receiving one or more neutral patient memories that do not evoke any of the plurality of positive emotions, such that providing the plurality of stimuli at step (i) comprises randomly providing one or more control stimuli based on the one or more neutral patient memories.

5. The method of claim 1, wherein providing the second exposure to one or more of the plurality of stimuli to the patient at step (iii) comprises providing a second exposure to the one or more stimuli that evoke the greatest measured brain activity amongst the plurality of stimuli.

6. The method of claim 1, wherein the graphical visualization corresponds and is linked in real-time to a region of activity in the brain, such that the graphical visualization automatically updates based on the emotional intensity of the given emotion associated with the one or more stimuli.

7. The method of claim 1, wherein the second exposure in step (v) comprises providing the one or more stimuli to the patient while displaying the graphical visualization and/or subsequent to displaying the graphical visualization.

8. The method of claim 1, wherein providing the one or more stimuli to the patient intermittently for the first period in step (vi) comprises providing the one or more stimuli to the patient at home, every three days for a period of two weeks.

9. The method of claim 1, wherein measuring the brain activity comprises a 7 Tesla (T) functional magnetic resonance imaging (fMRI) session.

10. The method of claim 1, wherein the treatment regimen comprises four fMRI sessions, each fMRI session spaced apart by a two-week at-home treatment session.

11. A method for treating a neurological deficiency in a patient, the method performed by a system comprising one or more processors, the method comprising:
providing to the patient a plurality of stimuli based on a plurality of positive memories of the patient;
while providing the plurality of stimuli, measuring brain activity of the patient by collecting data indicating one or more regions of activity in the brain and an associated intensity of brain activity in the one or more regions; and
displaying to the patient a graphical visualization representative of a plurality of positive emotions, wherein each emotion is assigned a color, and an intensity of the color in the graphical visualization indicates an emotional intensity of a given emotion that is associated with the one or more stimuli.

12. The method of claim 11, wherein the graphical visualization corresponds and is linked in real-time to a region of activity in the brain, such that the graphical visualization automatically updates based on the emotional intensity of the given emotion associated with the one or more stimuli.

13. The method of claim 11, wherein providing the plurality of stimuli comprises randomly providing one or more control stimuli based on one or more neutral patient memories.

14. The method of claim 11, wherein measuring the brain activity comprises a 7 Tesla (T) functional magnetic resonance imaging (fMRI) session.

15. The method of claim 11, comprising further providing one or more of the plurality of stimuli to the patient while displaying the graphical visualization and/or subsequent to displaying the graphical visualization.

16. The method of claim 11, wherein further providing the one or more of the plurality of stimuli to the patient comprises providing the one or more stimuli that evoke the greatest measured brain activity amongst the plurality of stimuli.

17. A system comprising one or more processors and memory storing instructions configured to be executed by the one or more processors to cause the system to:
provide to the patient a plurality of stimuli based on a plurality of positive memories of the patient;
while providing the plurality of stimuli, measure brain activity of the patient by collecting data indicating one or more regions of activity in the brain and an associated intensity of brain activity in the one or more regions; and
display to the patient a graphical visualization representative of a plurality of positive emotions, wherein each emotion is assigned a color, and an intensity of the color in the graphical visualization indicates an emotional intensity of a given emotion that is associated with the one or more stimuli.
